(19) United States
(12) Patent Application Publication
(10) Pub. No.: US 2026/0171248 A1
(43) Pub. Date: Jun. 18, 2026

(54) METHOD AND SYSTEMS FOR OPTIMIZING DIAGNOSTIC PLATFORM WORKFLOW USING ARTIFICIAL INTELLIGENCE MODELS

(71) Applicant: KONINKLIJKE PHILIPS N.V., EINDHOVEN (NL)

(72) Inventors: MAN CHING CHEUNG, Cambridge, MA (US); JON KAMERMAN, Andover, MA (US)

(21) Appl. No.: 19/411,606

(22) Filed: Dec. 8, 2025

Related U.S. Application Data
(60) Provisional application No. 63/734,253, filed on Dec. 16, 2024.

(51) Int. Cl. G16H 50/20; G06F 21/62; G16H 10/60

(52) U.S. Cl. CPC G16H 50/20; G06F 21/6245; G16H 10/60

(57) ABSTRACT

A diagnostic platform method (100), comprising: receiving (120), from one or more data sources, an identification of a plurality of patients; exporting (130) data from the received identification of the plurality of patients; providing (140) the exported data to each of a plurality of different machine learning algorithms; receiving (150) the generated analysis output from each of the plurality of different machine learning algorithms; analyzing (160) the provided data by each of the plurality of different machine learning algorithms to generate analysis output; and displaying (170), via a user interface of the diagnostic platform, the generated analysis output from each of the plurality of different machine learning algorithms.

### CROSS-REFERENCE TO RELATED APPLICATIONS

This patent application claims the priority benefit under 35 U.S.C. § 119(e) of U.S. Provisional Application No. 63/734,253, filed on Dec. 16, 2024, the contents of which are herein incorporated by reference.

### FIELD OF THE DISCLOSURE

The present disclosure is directed generally to methods and systems for managing artificial intelligence-integrated diagnostic analysis within a clinical workflow.

### BACKGROUND

Artificial intelligence (AI) models such as large-language models (LLMs) are a versatile class of deep learning neural networks, capable of retrieving and recombining data from immensely large repositories and organizing results into human-comprehensible answers or output. University hospitals, for example, are at the cutting edge of medical innovation and improving patient lives, and are beginning to utilize AI models for these purposes. These AI models come from many different sources, including both internal and external sources. For example, many third parties offer AI models that can be utilized by hospitals or other caregiving facilities.

One challenge with using third-party AI models is that vendors commercialize these algorithms, forcing hospitals into the role of consumers to derive insights from their own data. Another challenge is that AI models are often used to analyze primary physiological data that may include protected health information about patients, which can lead to data privacy and security issues.

### SUMMARY OF THE DISCLOSURE

There is thus a need for methods and systems that protect patient data while also integrating AI algorithms into clinical workflows.

Various embodiments and implementations are directed to artificial intelligence-integrated diagnostic methods and systems. A diagnostic platform receives, from one or more data sources, an identification of a plurality of patients. The diagnostic platform prepares and/or exports data from the received identification of the plurality of patients, which is then utilized in downstream steps of the method. The diagnostic platform provides the exported data to each of a plurality of different machine learning algorithms, which analyzes the provided data to generate analysis output. The system receives the generated analysis output from each of the plurality of different machine learning algorithms, and displays, via a user interface of the diagnostic platform, the generated analysis output from each of the plurality of different machine learning algorithms.

According to an aspect, a diagnostic platform method is provided. The method includes: (i) receiving, from one or more data sources, matched data identified for a plurality of patients; (ii) exporting data from the received matched data identified for the plurality of patients; (iii) removing protected health information and creating new protected health information to replace the protected health information; (iv) providing the exported data to each of a plurality of different machine learning algorithms; (v) analyzing the provided data by each of the plurality of different machine learning algorithms to generate analysis output for each of the identified plurality of patients; (vi) receiving the generated analysis output from each of the plurality of different machine learning algorithms; and (vii) displaying, via a user interface of the diagnostic platform, the generated analysis output from each of the plurality of different machine learning algorithms, identified and matched to the plurality of patients.

In accordance with an embodiment, receiving, from one or more data sources, information about a subject comprises receiving a list of patients and associated data matched to each subject. In accordance with an embodiment, the patients in the list of patients are identified by a medical record number or other unique identifier.

In accordance with an embodiment, the plurality of different machine learning algorithms are hosted within the diagnostic platform.

In accordance with an embodiment, one or more of the plurality of different machine learning algorithms is hosted by a third-party.

In accordance with an embodiment, one or more of the plurality of different machine learning algorithms is hosted on-premise, in a data center controlled by the first-party, in a data center controlled by a third party, or in any combination thereof.

In accordance with an embodiment, the method further includes removing protected health information from the exported data before providing the exported data to one or more of the plurality of different machine learning algorithms.

In accordance with an embodiment, the method further includes encrypting protected health information within the exported data before providing the exported data to one or more of the plurality of different machine learning algorithms.

In accordance with an embodiment, the method further includes removing protected health information from the exported data, creating multiple copies of the exported data, creating simulated protected health information, and creating a unique version of the exported data with different simulated protected health information, before encrypting protected health information and providing all copies of exported data to one or more of the plurality of different machine learning algorithms.

In accordance with an embodiment, the method further includes training, using the exported data, one or more machine learning algorithms.

In accordance with an embodiment, one or more of the plurality of different machine learning algorithms are accessed by the system via an API.

According to another aspect is a diagnostic platform system. The diagnostic platform system comprises: a processor configured to: (i) receive, from one or more data sources, matched data identified for a plurality of patients; (ii) export data from the received matched data identified for the plurality of patients; (iii) remove protected health information and create new protected health information data for each matched data; (iv) provide the exported data to each of a plurality of different machine learning algorithms; (v) analyze the provided data by each of the plurality of different machine learning algorithms and generate analysis output for each of the identified plurality of patients; ; (vi) receive the generated analysis output from each of the plurality of different machine learning algorithms; and a user interface configured to display the generated analysis output from each of the plurality of different machine learning algorithms.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. The figures showing features and ways of implementing various embodiments and are not to be construed as being limiting to other possible embodiments falling within the scope of the attached claims. Also, the drawings are not necessarily to scale, emphasis instead generally being placed upon illustrating the principles of the various embodiments.

FIG. 1 is a flowchart of a diagnostic platform method, in accordance with an embodiment.

FIG. 2 is a schematic representation of a diagnostic platform, in accordance with an embodiment.

FIG. 3 is a flowchart of a diagnostic platform method, in accordance with an embodiment.

FIG. 4 is a schematic representation of a user interface of a diagnostic platform, in accordance with an embodiment.

FIG. 5A is a flowchart of a diagnostic platform method, in accordance with an embodiment.

FIG. 5B is a flowchart of a diagnostic platform method, in accordance with an embodiment.

FIG. 6 is a flowchart of a diagnostic platform method, in accordance with an embodiment.

### DETAILED DESCRIPTION OF EMBODIMENTS

The present disclosure describes various embodiments of artificial intelligence-integrated diagnostic platforms and methods. More generally, Applicant has recognized and appreciated that it would be beneficial to provide a method and system to protect patient data while also integrating AI algorithms into clinical workflows. A diagnostic platform receives, from one or more data sources, an identification of a plurality of patients. The diagnostic platform prepares and/or exports data from the received identification of the plurality of patients, which is then utilized in downstream steps of the method. The diagnostic platform provides the exported data to each of a plurality of different machine learning algorithms, which analyzes the provided data to generate analysis output. The system receives the generated analysis output from each of the plurality of different machine learning algorithms, and displays, via a user interface of the diagnostic platform, the generated analysis output from each of the plurality of different machine learning algorithms.

The embodiments and implementations disclosed or otherwise envisioned herein can be utilized with any diagnostic platform or other platform utilized by a clinician to review information relevant to a subject or patient. For example, one application of the embodiments and implementations herein is to improve systems such as, e.g., the Philips® IntelliSpace® line of diagnostic and reporting tools (manufactured by Koninklijke Philips, N.V.), among many other products. However, the disclosure is not limited to these devices or systems, and thus the disclosure and embodiments disclosed herein can encompass any artificial intelligence-integrated method, device, or system.

According to an embodiment, the methods and systems described or otherwise envisioned herein utilize an artificial intelligence-integrated diagnostic platform to provide users of diagnostic platforms with integrated tools, specifically to help manage AI models—including those created by physicians or other users—and the wealth of insights. Thus, the methods and systems described or otherwise envisioned herein bring transparency, ownership, and integration of AI research and development into the clinic.

According to an embodiment, from a delivery perspective, the systems and methods described or otherwise envisioned herein provide access to data that is not exclusively within the diagnostic domain of the platform (for example, making data from PACS and echo available in an ECG platform), provide tooling to find and export specific patient data, provide a method to load and manage AI models, and enable AI results in the clinical workflow. Further, the systems and methods described or otherwise envisioned herein can be used to separate the viewing and editing of results or patient data in a diagnostic platform, from the machine learning algorithms used in or with the diagnostic platform.

According to an embodiment, the systems and methods described or otherwise envisioned herein comprise an options-based architecture with a clinical search component, whereby clinicians can find cohorts based on, for example, ECG measurements and diagnostic finding statements. One feature of the platform is to export patient records (e.g., anonymized) for clinician or researchers to develop their own algorithms. Customers (e.g., hospitals) require or desire the ability to plug-in their own algorithms and run inference in the context of regular workflow, including for validation purposes.

While there are current products that can provide tools to analyze primary physiological data, the systems and methods described or otherwise envisioned herein provide a way to manage AI models generated by customers, integrate the AI models into their clinical workflow, and deal with the insights derived from the deluge of AI vendors and models. Thus, the systems and methods described or otherwise envisioned herein provides customers with control over the scope of the AI models available in the current and future marketplace. There is thus an end-to-end solution for cohort selection, including exporting integrated physiological data for training, a way to upload trained models and set data dependencies and permissions, a way to preprocess data for model inference (e.g., use of trained models), a way to select the AI model (from a third-party or from in-house), and then a way for clinicians to view the original patient record alongside model results, mark model accuracy, and where relevant, provide results from multiple AI models as part of an AI panel.

According to an embodiment, the systems and methods described or otherwise envisioned herein comprises an AI-model tool with which clinicians can identify a cohort of patients outside of a diagnostic platform and upload the cohort list. The system match the cohort list and pull data from a diagnostic platform. Then the system takes trained AI algorithms and provides them to the diagnostic platform. This is a plug-in approach, where models are provided by third-party vendors, clinician-researchers, and optionally other sources. The plug-in manager approach provides a way for these physicians to use insights derived from their models, into their workflow. Thus, the systems and methods described or otherwise envisioned herein provide a model management flow by first providing data export capabilities and then by providing a platform to host, manage, and use their models. Over time, this develops into an AI model marketplace.

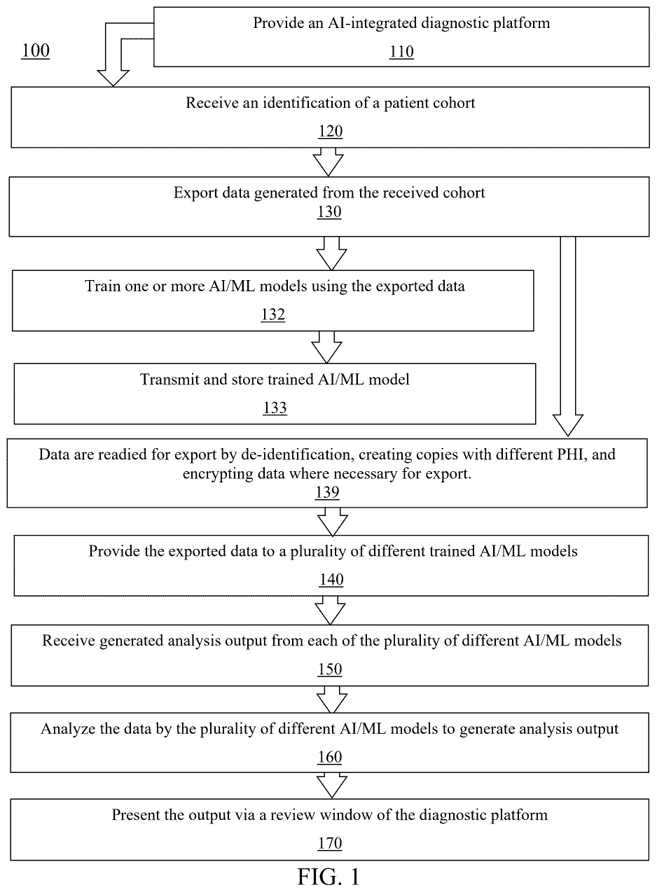

Referring to FIG. 1, in one embodiment, is a flowchart of a method 100 using a diagnostic platform. The methods described in connection with the figures are provided as examples only, and shall be understood not to limit the scope of the disclosure. The diagnostic platform can be any of the devices or systems described or otherwise envisioned herein. The diagnostic platform can be a single device or system, or can be multiple different devices or systems, or can be multiple devices or systems located proximally (e.g., an on-premise system) or distally (e.g., a cloud service provider).

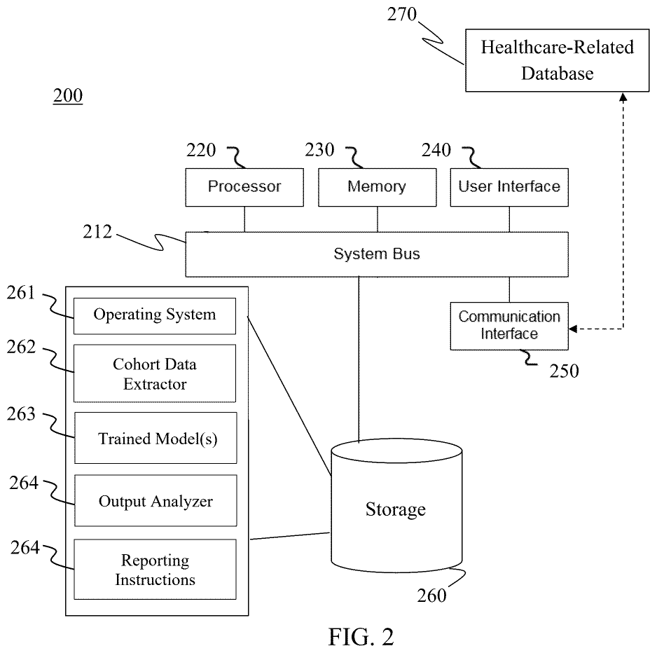

At step 110 of the method, a diagnostic platform 200 is provided. Referring to an embodiment of a diagnostic platform 200 as depicted in FIG. 2, for example, the system comprises one or more of a processor 220, memory 230, user interface 240, communications interface 250, and storage 260, interconnected via one or more system buses 212. It will be understood that FIG. 2 constitutes, in some respects, an abstraction and that the actual organization of the components of the diagnostic platform 200 may be different and more complex than illustrated. Additionally, diagnostic platform 200 can be any of the devices described or otherwise envisioned herein. Other elements and components of the diagnostic platform 200 are disclosed and/or envisioned elsewhere herein.

According to an embodiment, the diagnostic platform 200 comprises or is in direct or indirect communication with a healthcare-related database 270, such as for example an electronic medical record (EMR) database or system 270, comprising health data or other information for a plurality of patients or subjects. The health data can be any information about the plurality of patients or subjects. According to an embodiment, the information comprises one or more of demographic information about the patient, a diagnosis for the patient, medical history of the patient such as treatment information, lab or test results, and/or any other information. For example, demographic information may comprise information about the patient such as name, age, body mass index (BMI), and any other demographic information. The diagnosis for the patient may be any information about a medical diagnosis for the patient, historical and/or current. The medical history of the patient may be any historical admittance or discharge information, historical treatment information, historical diagnosis information, historical exam or imaging information, and/or any other information (although in some embodiments, a patient's medical history may not be available). The lab or test results may be, for example, an analysis of blood gases, electrolytes, biomarkers, ECG results, echocardiogram results, ultrasound results, CT results, MRI results, and/or any other types of lab tests or testing. Many other forms and types of patient health data are possible.

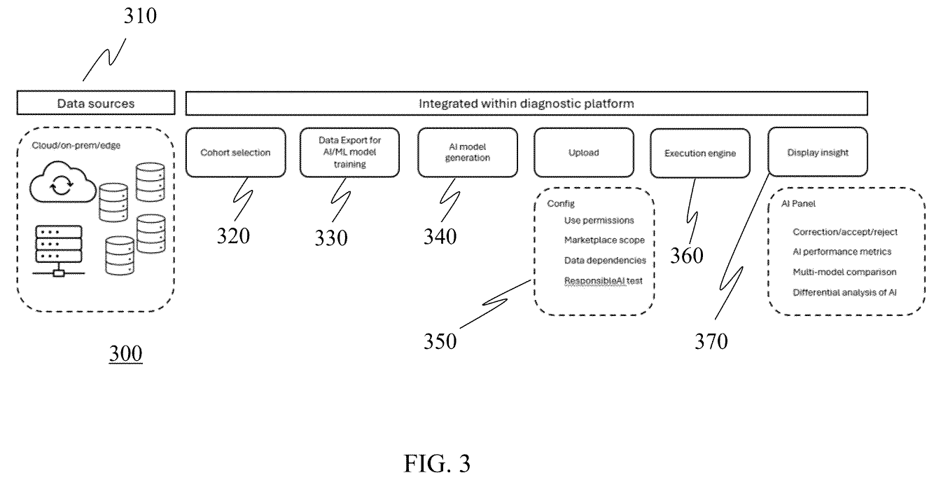

At step 120 of the method, the diagnostic platform receives identified and matched data for a plurality of patients, from one or more data sources. For example, referring to FIG. 3 is a schematic representation 300 of a method for analysis by a diagnostic platform. At 310, the diagnostic platform comprises or is in local and/or remote communication with one or more data sources comprising patient identification and/or information, as described or otherwise envisioned herein. At 320, there is a cohort selection (e.g., an identification of a plurality of patients from one or more data sources).

The cohort—also known as the plurality of patients—can be identified or selected in a variety of different ways. According to an embodiment, a clinician or other user can create an externally-derived patient list that is uploaded to the diagnostic platform. Alternatively, the diagnostic platform can comprise an internal search capability allowing clinicians want to select the patient cohort from within the diagnostic platform. Additionally, the diagnostic platform may comprise the ability to choose whether some patient identifiers are or are not exported. For example, medical record numbers (MRNs), data of study, age (e.g., by brackets), and gender are possible filters. Export criteria can be defined by the clinician or hospital based research, optionally in conjunction with uses allowed by the patient. According to an embodiment, when the cohort is selected within the diagnostic platform, no MRN may be released. According to an embodiment, a user interface of the diagnostic platform can comprise the ability filter and search by all available data elements. The data elements for export will be defined by the user.

According to an embodiment, the cohort can be selected by a clinician uploading a list of patients defined by protected health information (PHI), MRN, or other definition. The cohort selection tool, which can be a component of the diagnostic platform, can then search for the patients and export data as defined by the clinician.

At step 130, the system exports data from the received identification of the plurality of patients. This can be done in a variety of different ways. According to one embodiment, the data as defined by the clinician can be exported into various formats, including but not limited to comma-delimited format, spreadsheets, JSON, ODBC protocol, and others. According to another embodiment, the data is exported into a machine learning/artificial intelligence (ML/AI) plugin module, where the data can be directly used by a training system. In this case, the diagnostic platform may comprise an AI Orchestrator. The AI Orchestrator does not have to provide the data to the system, but can instead provide a pathway to a training system. The AI Orchestrator can also host the AI training platform (e.g., embedding a Python instance into the platform). According to yet another embodiment, embeddings are created from the data to be exported, such that the data is ready for advanced deep-learning techniques. Referring again to FIG. 3, at 330 there is export of the data derived from the identified cohort.

According to an embodiment, the system comprises a data orchestrator to cache relevant out-of-modality data. This orchestrator can work in a generative mode or by query. The data orchestrator is configured to utilize prior patient data, and the control of this orchestrator can be integrated into the overall AI orchestrator.

For a query approach, the data orchestrator may comprise a transformer-based model architecture to create embeddings of diagnostic information. The model can be fine-tuned so that similarity is based on semantic and contextual meaning. The model can be extended to include out of modality data. For example, an ECG diagnosis of “right bundle branch block” can be given additional context, if an image-based modality (echo, MRI, CT) measurement of ejection fraction is available. Abnormally low EF and an observation of RBBB is indicative on progressive heart failure. Thus, having a model that can be paired so that it can bring related information can be useful.

For a generative approach, the data orchestrator may use large-language models or other ML/AI models to summarize prior data. In this way, the model can be trained on a medical corpus and then fine-tuned on the findings and statements available from the diagnostic platforms. The system would also require access to patient information. The diagnostic findings are thus prompts which can then trigger the LLM to return results. In this case, only that patient's data can be used for context such that patient specific results are returned.

For data export, such as for AI/ML training purposes, the system may comprise a data management module. The AI/ML data tool can take in a list of MRNs. For example, the system can use ECGs associated with the MRNs which will have waveforms and measurements exported. Another approach is to export data by using search filters, in the diagnostic platform, to identify the cohort of interest. The diagnostic platform reporting tool may thus comprise the ability to search by measurements, patient demographics, and finding codes.

At optional step 132 of the method, an AI model or other machine-learning algorithm can be trained using the exported data. The model can be any model that can be trained to utilize the input to generate the output, as described or otherwise envisioned herein. For example, the model can be a neural network or other trained machine learning model, for example a convolutional neural network (CNN) or a transformer network or other neural network. Thus, according to an embodiment, the diagnostic platform 200 comprises or is in local and/or remote communication with a model that receives the input data (e.g., the exported data) and outputs the various output as described or otherwise envisioned herein. The AI model or other machine-learning algorithm can be trained within the AI Orchestrator platform, or in a platform linked to the AI Orchestrator, or elsewhere performed by the researcher. The AI model or other machine-learning algorithm is trained using any of a variety of methods to train a model. Referring again to FIG. 3, at 340 there is AI model generation, via training of the AI model or other machine-learning algorithm.

At optional step 133 of the method, if a model from step 132 is trained and the clinician sees fit to include the model for general use, the model can be transmitted, stored, and added as one of the plurality of different machine learning algorithms used in step 140. Specific model use rights information can be stored with the model. Referring again to FIG. 2, at 263 there is “Trained Model(s)”, in which models can be stored to become one of the plurality of machine learning algorithms. Referring again to FIG. 3, at 350 there is “Upload”, by which the model and use permissions are stored.

At step 139 of the method, the diagnostic platform provides a way to remove protected health information and obscure protected health information before sending to each of a plurality of different machine learning algorithms. This can be accomplished in a variety of different ways. According to an embodiment, the protected health information can be removed. According to an embodiment, protected health information that is needed for some of the plurality of machine learning algorithms may be encrypted. According to an embodiment, the protected health information can be removed, sets of protected health information can be randomly generated, and multiple copies of the data with different sets of protected health information can be created. Multiple copies of the exported data can be provided to each of a plurality of different machine learning algorithms

At step 140 of the method, the diagnostic platform provides the exported data to each of a plurality of different machine learning algorithms. This can be accomplished in a variety of different ways. According to an embodiment, the system comprises a management module for uploading, storing, and selecting active AI models. The module may be utilized to provide the platform to clinician-researchers. According to an embodiment, AI/ML models trained by customers can be uploaded to the system, such as via the management module, at step 133.

According to an embodiment, the management module (or any other component of the diagnostic platform) can access third-party (e.g., vendor) AI/ML models via an API or other method. According to an embodiment, input data dependencies can be part of the AI-model usage specifications. This is so that data availability is assessed (for example, it may be that findings from a modality are needed, or some other specific data field), due to the system storing and connecting to data outside of the diagnostic platform. Access rights can also be managed and specified (by use or by personnel).

At step 150 of the method, the diagnostic platform receives the generated analysis output from each of the plurality of different machine learning algorithms. At step 160 of the method, the diagnostic platform analyzes the provided data using each of the plurality of different machine learning algorithms to generate analysis output, as described or otherwise envisioned herein. If step 139 included sending multiple copies of data with randomized protected health information, step 160 will receive the generated analysis output and select the correct output based on identification of the patient subject.

According to an embodiment, the AI orchestrator assesses the context of the AI model and required data, and mode of function. Generally, insights that use out of modality, pre-existing data are concerned with surfacing the correct information. According to an embodiment, specific diagnostic algorithms that operate on the current piece of physiological data will display on the primary viewer (for example, on the ECG, or Echo, or MRI, etc.). There can be an associated explainer, such as features and significance, or highlighting of informative areas on the data. According to an embodiment, results, geared for uses downstream of the current diagnoses, appear here. These can include summaries sent to patients, referring physicians, or enter into patient record as progress notes. These summaries and conclusions are of immense value, representing the best judgment and observations of the clinician, and thus is not simply diagnostic statements.

According to an embodiment, for AI/ML-based diagnostic algorithms and context generation, various vendors may provide AI function (i.e., inference) in the cloud. As a part of AI orchestrator, the system provides a safer way of sending raw data to engage with remote based AI models in two ways. First, by scrubbing health data of protected health information and then adding randomized parameters for PHI. After obtaining results for multiple inferences, the system can construct the original finding since there can be a data privacy module to resolve the true result for the patient. Further, use of AI in diagnostics can be approved or disallowed by the patient. Second, the system can encrypt the necessary information using public-key encryption, where vendors who are onboarded to the AI orchestrator platform can obtain a specific decryption key. The system can also use combination of these two approaches. In this way, the system provides only the bare minimum of information for AI/ML inference, and there can be additional encryption for data-in-flight beyond that of SSL protocols.

According to an embodiment, in order to use a model within AI Orchestrator, the model will be uploaded into the Orchestrator. The AI model creator can also upload a set of input requirements (such as the dimensions of the data, the format of the data, the needed data elements, and so forth). Descriptions of the model can be tracked by the AI Orchestrator, so that the clinician can identify the model and revision of the model being used. Third-party models can also be listed, along with required data. In this way, available models will be controlled by the hospital administrators.

According to an embodiment, the system may comprise an execution engine, for executing the plurality of AI/ML models using the exported data. The diagnostic platform may comprise UI module which can be a component of the diagnostic platform, so that the clinician can define which module to use, which sets of data to send to the model, and so on. The UI module can have a run inference button as well, to implement execution.

According to an embodiment, the available AI/ML models will populate from a list of uploaded models, or defined models licensed or purchased from various vendors. This includes hospital created or third-party AI/ML models. Data will also be defined and associated with each model so that the necessary data are sent to the AI/ML model. In addition, patient data authorization rights will also be checked so that only with proper patient consent and use authorization will patients'health data be sent to AI models. For added PHI protection, the engine can also model required identifying and quasi-identifying data as well and package data for remote or cloud-based AI inference.

According to an embodiment, there can be a variety of mechanisms for sending data to AI providers (e.g., outside of the AI Orchestrator). One current architecture is to use RESTful APIs, where data can be sent and received, along with a payload. Data payloads can be encrypted, with decryption keys provided to partner vendors to further enhance security of transmitting patient health data.

Referring again to FIG. 3, at 350 there is upload of one or more of the plurality of different AI/ML algorithms to the system. At 360, there is implementation of the AI/ML algorithms by the execution engine.

At step 170 of the method, the diagnostic platform displays, via a user interface, the generated analysis output from each of the plurality of different machine learning algorithms. For example, the generated analysis output can be presented to the clinician via a review window or interface 240 of the diagnostic platform 200. The generated analysis output can be any of the information as described or otherwise envisioned herein. The system may provide the information to a user via any mechanism, including but not limited to the visual display. The information may be communicated by wired and/or wireless communication to another device. For example, the system may communicate the information to a monitor, screen, mobile phone, computer, laptop, wearable device, and/or any other device configured to allow display and/or other communication of the information in the diagnostic platform.

According to an embodiment, there can be multiple output panels on the user interface to display the generated analysis output. For example, according to one embodiment, there can be at least two panels, a first panel summarizing data from prior labs and studies, and a second one integrating and summarizing results that include the current study. An output panel can comprise multiple options for documenting and correcting the results. It can include tailoring the summary text for audience, or parsing into structured codes, or further streamlined for billing, or rejected. The clinician can edit and is in full control of how to handle the information.

According to an embodiment, upon receiving AI/ML-generated analysis output, a processing engine can format the results for display in various UI modules. The AI Orchestrator can provide RESTful APIs and leverage public-key encryption protocols to receive AI inference results from partners. The UI modules can be integrated in various platforms, including diagnostic platforms.

According to an embodiment, based on the proliferation of AI models in the healthcare space, the AI Orchestrator can also compare and contrast results from multiple vendors, providing a meta-scoring where multiple AI models can be assessed for differences and overlaps in diagnoses. The AI Orchestrator can also rank the results by their confidence scores (e.g., calculated by the AI models). The mix of AI models being used will be defined by the user (or by hospital administrators), such that only results desired by the user will be presented.

According to an embodiment, the system may comprise an explainer sub-module such that AI-calculated features can be presented to the user. For example, deep-learning models may return SHAP results which highlight features that can explain a significant portion of the variance. These results can be used to annotate the source physiological data to visually highlight the prominent contributors to the AI result.

According to an embodiment, when multiple AI results are returned, a visualization (for example, a spider-plot) can be created to visually plot and quantify similarity of results, further helping the user find where AI models achieve consensus or divergence. In this case, a non-AI tool may be used to identify overlap, or an AI tool can be trained to summarize and edit down the information being returned by the models.

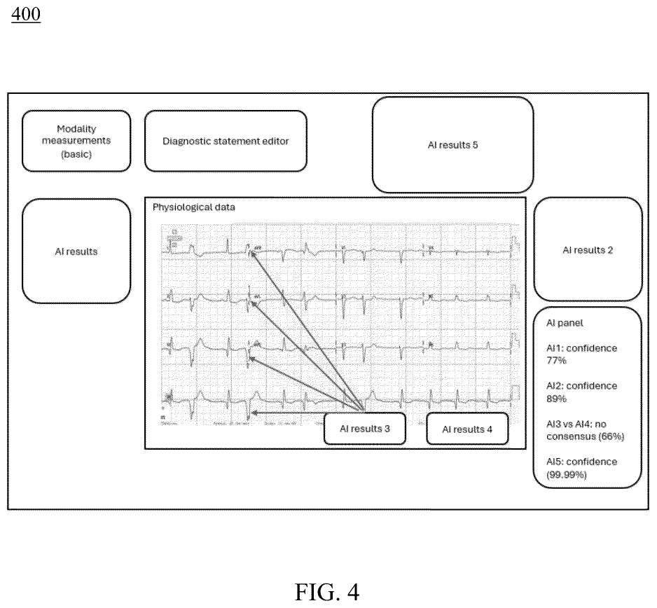

Referring again to FIG. 3, at 370 there is a display of insights, as described or otherwise envisioned herein. For example, referring to FIG. 4 is a schematic representation 400 of a visual display, in accordance with an embodiment. The visual display can include one or more of modality measurements, a diagnostic statement editor, AI/ML results, and an AI/ML panel, along with patient results.

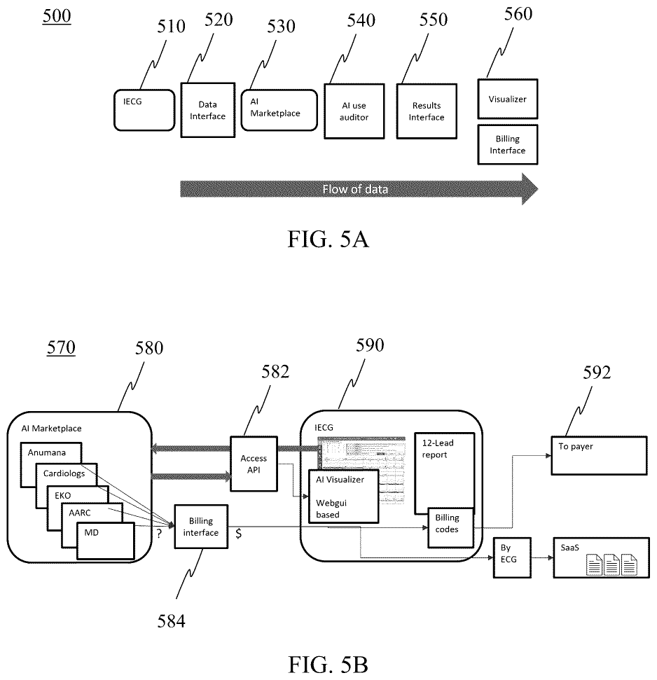

Referring to FIG. 5A, in one embodiment, is a schematic representation 500 of a system or method for using a diagnostic platform as described or otherwise envisioned herein, showing the flow of data (from left to right). The system and method begins with a diagnostic platform 510 (which in this non-limiting example is an ECG platform). The system comprises a data interface 520, which receives or obtains a list of a plurality of patients and associated health data for which information will be analyzed. The system also comprises an AI/ML marketplace 530, which comprises and contains a plurality of AI/ML algorithms. These algorithms can be entirely in-house, third-party, or a combination of the two. The system comprises an AI use auditor 540 which monitors or manages analysis of the patient data by the plurality of AI/ML algorithms, and the results interface 550 formats and/or otherwise provides the output of the analysis by the AI/ML algorithms. For example, the output can be presented via a user interface 560, which can include a visualizer and - where billing is required for use of an AI/ML algorithm—a billing interface to send the resulting information to the billing and revenue department at a hospital.

Referring to FIG. 5B, in one embodiment, is a schematic representation 570 of a system or method for using a diagnostic platform as described or otherwise envisioned herein, showing interaction of the system with an AI Marketplace 580. The AI Marketplace 580 comprises a plurality of AI/ML algorithms which can be accessed by a user of system 200. According to an embodiment, a user of system 590 accesses the AI Marketplace via an Access API 582. The system can also comprise a Billing Interface 584, where billing is required for use of an AI/ML algorithm, in order to enable the billing.

The system 590 (which in this non-limiting example is an ECG platform) can include an AI Visualizer that allows interaction with the plurality of AI/ML algorithms. This can also incorporate billing codes and other information to enable billing. The system can send data to a payer and/or to other outside sources at 592.

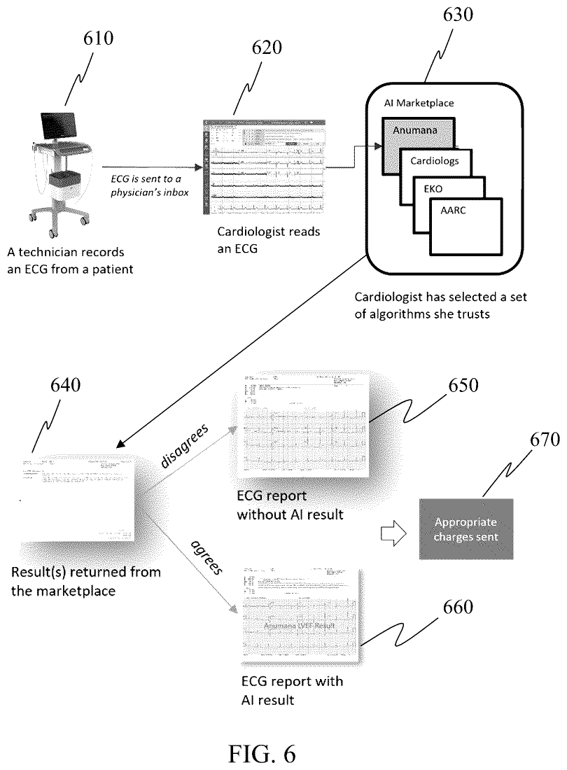

Referring to FIG. 6, in one embodiment, is a schematic representation 600 of a system or method for using a diagnostic platform as described or otherwise envisioned herein. At 610, a clinician obtains information from a patient (which in this non-limiting example is ECG data obtained using an ECG platform). The information is sent or otherwise obtained for review by a clinician (e.g., a cardiologist) at 620. The cardiologist, using the analysis system as described or otherwise envisioned herein, interacts with a plurality of AI/ML algorithms (“a set of algorithms that the clinician trusts”) at 630. At 640, the data is analyzed by the plurality of AI/ML algorithms to generate a set of output. The clinician can agree at 660 with the results, or can disagree at 650 with the results. At 670 appropriate charges are sent, based on the billing for use of the AI/ML algorithm(s).

Referring again to FIG. 2 is a schematic representation of a diagnostic platform 200. System 200 may be any of the devices or systems described or otherwise envisioned herein, and may comprise any of the components described or otherwise envisioned herein. It will be understood that FIG. 2 constitutes, in some respects, an abstraction and that the actual organization of the components of system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, system 200 may also comprise or be in direct or indirect communication with an electronic medical record system and/or an electronic medical records (EMR) database from which the information about patients may be obtained or received. For example, EMR database may comprise information about previous results, diagnostic information, or any other information about a patient. According to an embodiment, the electronic medical record system may be a local or remote database and is in direct and/or indirect communication with system 200. Thus, according to an embodiment, the system comprises an electronic medical record database or system.

According to an embodiment, storage 260 of system 200 may store one or more algorithms, modules, and/or instructions to carry out one or more functions or steps of the methods described or otherwise envisioned herein. For example, storage 260 may comprise, among other instructions or data, a cohort data extractor 262, one or more trained AI/ML models 263, output analyzer 264, and/or reporting instructions 265.

According to an embodiment, cohort data extractor 262 directs the system to extract data from the information received from the one or more data sources and/or input from the clinician about the plurality of patients. The cohort data extractor may, for example, be implemented as a module or component of the processor 220 of the system, such as by software or an algorithm. However, the system is not limited to the implementation of the cohort data extractor, and thus other processes for extracting data are possible. According to an embodiment, the cohort data extractor extracts data from plurality of patients identified in a cohort. The data can comprise one or more modalities, such as text, waveforms, images, and more. The extracted data can be transformed into embeddings for subsequent processing.

According to an embodiment, the one or more trained AI/ML models 263 are trained to analyze the received input, including but not limited to the generated cohort data, to generate analysis output about the set of results. The one or more trained AI/ML models 263 can be any model that can be trained to utilize the input to generate the output, as described or otherwise envisioned herein. For example, the model can be a neural network or other trained machine learning model, for example a convolutional neural network (CNN) or a transformer network or other neural network. Thus, according to an embodiment, the system 200 comprises one or more trained AI/ML models 263 that receives the input data and outputs the diagnostic output, as described or otherwise envisioned herein. According to an embodiment, the system may comprise an execution engine, for executing the plurality of AI/ML models using the exported data. The diagnostic platform may comprise UI module which can be a component of the diagnostic platform, so that the clinician can define which module to use, which sets of data to send to the model, and so on. The UI module can have a run inference button as well, to implement execution.

According to an embodiment, output analyzer 264 is configured to receive the generated analysis output from each of the plurality of different machine learning algorithms. The output analyzer 264 can be further configured to process or format the analysis output received from each of the plurality of different machine learning algorithms.

According to an embodiment, reporting instructions 265 directs the system to provide the output of the system—such as the diagnostic output of the AI models—to a patient, clinician, or to another device or system. The provided output can be any of the information as described or otherwise envisioned herein. The system may provide the information to a user via any mechanism, including but not limited to a visual display. The information may be communicated by wired and/or wireless communication to another device. For example, the system may communicate the information to a monitor, screen, mobile phone, computer, laptop, wearable device, and/or any other device configured to allow display and/or other communication of the information. According to an embodiment, the system displays some or all of the output in the diagnostic platform, such as in a review window of the platform.

According to an embodiment, system 200 is configured to process many thousands or millions of datapoints in the input data used to train the one or more trained AI/ML models 263, where the model is generated or trained in-house. For example, generating a functional and skilled trained neural network encoder from a corpus of training data requires processing of millions of datapoints from input data and generated features. This can require millions or billions of calculations to generate a novel AI/ML model from those millions of datapoints and millions or billions of calculations. As a result, each trained model is novel and distinct based on the input data and parameters of the model, and thus improves the functioning of the system. Generating a functional and skilled trained model comprises a process with a volume of calculation and analysis that a human brain cannot accomplish in a lifetime, or multiple lifetimes.

Further, the methods and systems described or otherwise envisioned herein are specific methods or systems that improve diagnostic platform technology, rather than abstractly covering results without regard to a specific process or machinery for achieving those results. Here, the method and system for generating the diagnostic output by the one or more trained AI/ML models is a specific method and system that improves diagnostic platform technology. Specifically, the method and system comprises an improved diagnostic platform that generates and provides the diagnostic output in the review window of the diagnostic platform, and facilitates analysis by a plurality of different trained AI/ML models without having to interact with each model individually. This saves the clinician time and energy, and avoids distraction, thus also improving healthcare by the clinician. Prior art diagnostic platforms do not implement a plurality of trained AI/ML models within the review window of the platform, instead requiring the clinician leave the platform to engage with the plurality of trained AI/ML models. Thus, the results are generated using multiple different user interfaces, thereby requiring both increased clinician time/effort and computational time/effort. In contrast, the methods and systems described or otherwise envisioned herein implement the trained AI/ML models within the platform, which is more time- and computationally efficient. Thus, the processes described or otherwise envisioned herein improve the functionality of the diagnostic platform as it requires fewer user interfaces and less time or computational effort generate the diagnostic outputs. The methods and systems thus require specific, technological means—i.e., the cohort data extractor, the trained AI/ML models, the output analyzer—that in turn provides a technological improvement to the diagnostic platform. Thus, the methods and systems recite a technological solution to a technological problem.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A diagnostic platform method (100), comprising:
receiving (120), from one or more data sources, an identification of a plurality of patients, further comprising associated health data for the plurality of patients;
exporting (130) data from the received identification and associated health data of the plurality of patients;
providing (140) the exported data to each of a plurality of different machine learning algorithms;
receiving (150) the generated analysis output from each of the plurality of different machine learning algorithms;
analyzing (160) the provided data by each of the plurality of different machine learning algorithms to generate analysis output; and
displaying (170), via a user interface of the diagnostic platform, the generated analysis output from each of the plurality of different machine learning algorithms.

2. The method of claim 1, wherein receiving (120), from one or more data sources, information about a subject comprises receiving a list of patients.

3. The method of claim 2, wherein the patients in the list of patients are identified by a medical record number or other unique identifier.

4. The method of claim 1, wherein the plurality of different machine learning algorithms are hosted within the diagnostic platform.

5. The method of claim 1, wherein one or more of the plurality of different machine learning algorithms is hosted by a third-party.

6. The method of claim 1, further comprising removing protected health information from the exported data before providing the exported data to one or more of the plurality of different machine learning algorithms.

7. The method of claim 1, further comprising, before providing the exported data to one or more of the plurality of different machine learning algorithms: removing protected health information from the exported data, generating a set of random protected health information, creating multiple copies of the exported data each with a different set of protected health information.

8. The method of claim 1, further comprising encrypting protected health information within the exported data before providing the exported data to one or more of the plurality of different machine learning algorithms.

9. The method of claim 1, further comprising the step of training (132), using the exported data, one or more machine learning algorithms.

10. A diagnostic platform system (200), comprising:
a processor (220) configured to: (i) receive, from one or more data sources, an identification of a plurality of patients, further comprising associated health data for the plurality of patients; (ii) export data from the received identification and associated health data of the plurality of patients; (iii) provide the exported data to each of a plurality of different machine learning algorithms; (iv) receive the generated analysis output from each of the plurality of different machine learning algorithms; and (v) analyze the provided data by each of the plurality of different machine learning algorithms to generate analysis output; and
a user interface (240) configured to display the generated analysis output from each of the plurality of different machine learning algorithms.

11. The diagnostic platform system of claim 10, wherein receiving information about a subject comprises receiving a list of patients and their associated health data.

12. The diagnostic platform system of claim 10, wherein the plurality of different machine learning algorithms are hosted within the diagnostic platform.

13. The diagnostic platform system of claim 10, wherein one or more of the plurality of different machine learning algorithms is hosted by a third-party.

14. The diagnostic platform system of claim 10, wherein the processor is further configured to remove protected health information from the exported data before providing the exported data to one or more of the plurality of different machine learning algorithms, or wherein the processor is further configured to encrypt protected health information within the exported data before providing the exported data to one or more of the plurality of different machine learning algorithms.

15. The diagnostic platform system of claim 10, wherein the processor is further configured to, before providing the exported data to one or more of the plurality of different machine learning algorithms, remove protected health information from the exported data, generate a set of random protected health information, and create multiple copies of the exported data each with a different set of protected health information.
